(19) United States
(12) Patent Application Publication
(10) Pub. No.: US 2011/0177525 A1
(43) Pub. Date: Jul. 21, 2011

(54) ANTIBODIES AND METHODS OF DIAGNOSING DISEASES

(75) Inventors: Anthony P. Shuber, Mendon, MA (US); Cecilia Fernandez, Jamaica Plain, MA (US)

(73) Assignee: Predictive Biosciences, Inc., Lexington, MA (US)

(21) Appl. No.: 12/689,542

(22) Filed: Jan. 19, 2010

(51) Int. Cl. G01N 33/53; C07K 16/00; C12N 9/48

(52) U.S. Cl. 435/7.2; 530/387.1; 530/387.3; 530/389.1; 435/212; 436/501; 435/7.8

(57) ABSTRACT

The present invention generally relates to antibodies and use of these antibodies in diagnostic assays for various disease states, including cancer. In certain embodiments, the invention provides an isolated human or humanized antibody or functional fragment thereof including an antigen-binding region that is specific for an epitope on a protein, in which the epitope is specific to a tissue or body fluid and the epitope is indicative of a disease.

### FIELD OF THE INVENTION

The present invention generally relates to antibodies and use of these antibodies in diagnostic assays for various disease states, including cancer.

### BACKGROUND

Antibodies are proteins of the body's immune system that detect and neutralize foreign particles known as antigens. Antibodies bind to a very specific part of the antigen referred to as the epitope, and this binding is highly specific. Common diagnostic tools for various diseases take advantage of the specific interaction between an antibody and an antigen.

While there are panels of antigens that are linked to detection of many diseases, these diagnostic tools are frequently hampered by limitations. Most diagnostic tests are based on a concentration determination, i.e., there is a baseline level of antigen present in a sample from a patient and a disease state is indicated by antigen levels either above or below that baseline level. For example, antibody detection of prostate specific antigen (PSA) in the blood of men potentially afflicted with prostate cancer is a common diagnostic tool. The American Cancer Society reports that most men have a PSA concentration of about 0 ng/ml to about 4 ng/ml of blood while a PSA concentration of about 10 ng/ml of blood is indicative of a 50% chance of prostate cancer. A common limitation of this type of concentration-based diagnostic assay is the propensity for false positives, i.e., detection of a signal in a patient that does not have the disease. For example, PSA levels near or above 10 ng/ml of blood are frequently detected in patients that are not suffering from prostate cancer. This artificial elevation of PSA level is commonly observed in patients with non-cancerous afflictions of the prostate such as prostatitis or benign prostatic hyperplasia (Schröder F H. Recent Results Cancer Res. 181:173-82, 2009). Thus, there are serious limitations in antibody-based diagnostic tests that rely on concentrations of a marker above a baseline.

A further limitation in diagnostic tests is the difficulty of making conclusions as to which disease is indicated by the test results. For instance, carcinoembryonic antigen (CEA) is a glycoprotein involved in cell adhesion that is produced during human development. In healthy individuals, production of CEA terminates before birth. Research has shown that expression of this antigen in individuals after birth is indicative of certain forms of cancer. CEA is a reliable marker for cancer, yet it presents in a variety of different cancers, including colorectal carcinoma, gastric carcinoma, pancreatic carcinoma, lung carcinoma and breast carcinoma. (Rudden, R., Cancer Biology, Fourth Ed, Oxford University Press US, 2007). Therefore, this marker requires that the clinician undertake further tests to determine which particular cancer is afflicting a patient

Thus, there is a need for improved and more accurate methods for diagnosing diseases, such as cancer, in patients.

### SUMMARY

The invention generally relates to antibodies that may be used for disease diagnosis, and involves detection of epitopes that are specific to a tissue or body fluid and that are associated with a disease. The invention eliminates inaccuracies and false positives associated with concentration-based assays by providing for absolute detection of a disease state, i.e. the presence of a signal, regardless of quantity, is indicative of a disease state. Therefore, the invention allows a clinician to assay a sample and definitively determine if a patient presents with a particular disease based on the presence of an antigen having a particular epitope, rather than diagnosing based upon a concentration of a biomarker in the sample.

Aspects of the invention provide for antibodies or functional fragments of the antibodies with specificity for an epitope on a protein, in which the epitope is specific to a tissue or body fluid and the epitope is indicative of a disease state. In particular embodiments, the antibodies or functional fragments of the antibodies bind to an epitope of ADAM12, in which the epitope is specific to urine and indicative of a cancer, such as is bladder, ovarian, or breast cancer. Antibodies of the invention may be monoclonal or polyclonal.

Another aspect of the invention provides for a protein including an epitope, in which the epitope is specific to a tissue or body fluid and the epitope is indicative of a disease state, such as an ADAM12 protein including an epitope specific to urine, in which the epitope is indicative of a cancer, such as is bladder, ovarian, or breast.

Antibodies of the invention may be used for diagnosing a disease in a subject. In order to detect an antibody/antigen complex, the antibody may be detectably labeled. Assays may be performed to detect the epitope in which the labeled antibody directly contacts the epitope of an antigen. In other assays, the labeled antibody indirectly contacts the epitope, e.g., the labeled antibody binds an unlabeled antibody that is bound to the epitope of the antigen.

Antibodies of the invention can screen for any disease associated with presentation of a particular epitope in a tissue. In particular embodiments, the disease is cancer. Exemplary cancers include brain, kidney, liver, adrenal gland, bladder, cervix, breast, stomach, ovaries, esophagus, neck, head, skin, colon, rectum, prostate, pancreas, liver, lung, vagina, thyroid, sarcomas, glioblastomas, multiple myeloma, blood, and gastrointestinal.

Tissue or body fluid samples can be obtained by any method known in the art. For example, the tissue sample can be obtained from a biopsy. The body fluid sample can be any fluid from a mammalian source (e.g., human), such as urine, sputum, stool, blood, mucus, or saliva. The epitope can be on a protease. The protease can be a metalloproteinase such as ADAM12.

Particular antibodies of the invention are used for diagnosing a cancer in a subject. Such use involves obtaining a urine sample from a subject, and contacting an antibody to the urine sample to detect an ADAM12 epitope that is specific to urine, in which presence of the epitope is indicative of a cancer. In certain embodiments the cancer is bladder, ovarian, or breast cancer. The immunoassay may include either a polyclonal antibody, monoclonal antibody, or functional fragment of either.

### BRIEF DESCRIPTION OF THE DRAWINGS

FIG. 1 is a photograph of a gel showing results of a western blot using AbCam ab28225 polyclonal ADAM12 antibody against ADAM12 in different samples.

FIG. 2 is a photograph of a gel shown western analysis of controls and urine samples using sera from mouse 2 (left) and mouse 5 (right). The band of interest at ˜68 kDa is indicated by the arrow. RR CM=conditioned media from Roopali Roy, CM=conditioned media from Blue Sky Pilot 1, SF=soluble Fraction from Pilot 1, F1=Purified ADAM12 from soluble fraction, 170=bladder cancer urine, 249=breast cancer urine, and 251=breast cancer urine.

FIG. 3 panels A and B are graphs showing monoclonal antibody development. Panel A shows development for ELISA reactivity of mouse antisera to ADAM12 cell paste. Panel B shows development for ELISA reactivity of mouse antisera to ADAM12 CM.

FIG. 4 is a photograph of a gel shown western analysis of controls and urine samples using sera from mouse 2 (left) and mouse 5 (right). The band of interest at ˜68 kDa is indicated by the arrow. RR CM=conditioned media from Roopali Roy, CM=conditioned media from Blue Sky Pilot 1, SF=soluble Fraction from Pilot 1, F1=Purified ADAM12 from soluble fraction, Normal Pool=urine from 5 normal individuals, and Cancer Pool=urine from 10 individuals with bladder cancer.

FIG. 5 panels A-D are photographs of gels showing western analysis of four Hybridoma candidates against pools of cancer and normal urine samples. The bands of interest are indicated by arrows. The representative blots above illustrate the multiple isoforms of ADAM12 that are recognized by various individual antibodies, The antibodies in panels A and D recognize the ˜68 kDa form that has previously been reported as a marker of breast cancer states. The antibodies in panels B and C recognize the ˜80 kDa full-length form of ADAM12.

FIG. 6 panels A and B are graphs showing monoclonal antibody development for mouse #2. Panel A shows development for ELISA reactivity of mouse #2 antisera to ADAM12 cell paste. Panel B shows development for ELISA reactivity of mouse #2 antisera to ADAM12 CM.

### DETAILED DESCRIPTION

The present invention generally relates to antibodies and use of these antibodies in diagnostic assays for various disease states, including cancer. Aspects of the invention provide isolated human or humanized antibodies or functional fragments thereof including an antigen-binding region that is specific for an epitope on a protein, in which the epitope is specific to a tissue or body fluid and the epitope is indicative of a disease. In particular embodiments, the isolated human or humanized antibody or functional fragment thereof includes an antigen-binding region that is specific for ADAM12 present in urine and that is indicative of a cancer.

The term “antibody” as referred to herein includes whole antibodies and any antigen binding fragment (i.e., “antigen-binding portion”) or single chains of these. A naturally occurring “antibody” is a glycoprotein including at least two heavy (H) chains and two light (L) chains inter-connected by disulfide bonds. Each heavy chain includes a heavy chain variable region (abbreviated herein as V_{H}) and a heavy chain constant region. The heavy chain constant region includes three domains, CH1, CH2 and CH3. Each light chain includes a light chain variable region (abbreviated herein as V_{L}) and a light chain constant region. The light chain constant region includes one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to an antigen (e.g., ADAM12). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment including two Fab fragments linked by a disulfide bridge at the hinge region; a Fd fragment consisting of the V_{H} and CH1 domains; a Fv fragment consisting of the V_{L} and V_{H} domains of a single arm of an antibody; a FAb fragment (Ward et al., 1989 Nature 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

In a particular embodiment, the antibody is an isolated human or humanized antibody or functional fragment thereof including an antigen-binding region that is specific for target protein ADAM12 present in urine and indicative of a caner, in which the antibody or functional fragment thereof binds to ADAM12. The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

As used herein, an antibody that “binds an epitope that is specific to the tissue or body fluid and that is indicative of a disease state” is intended to refer to an antibody that binds to the epitope with a K_{D} of 5×10⁻⁹ M or less, 2×10⁻⁹ M or less, or 1×10⁻¹⁰ M or less. For example, the antibody is monoclonal or polyclonal. The terms “monoclonal antibody” or “monoclonal antibody composition” as used herein refer to a preparation of antibody molecules of single molecular composition. A monoclonal antibody composition displays a single binding specificity and affinity for the epitope that is specific to the tissue or body fluid and that is indicative of a disease state. The antibody is an IgM, IgE, IgG such as IgG1 or IgG4.

Also useful is an antibody that is a recombinant antibody. The term “recombinant human antibody”, as used herein, includes all antibodies that are prepared, expressed, created or isolated by recombinant means, such as antibodies isolated from an animal (e.g., a mouse). Mammalian host cells for expressing the recombinant antibodies used in the methods herein include Chinese Hamster Ovary (CHO cells) including dhfr-CHO cells, described in Urlaub and Chasin, Proc. Natl. Acad. Sci. USA 77:4216-4220, 1980 used with a DH FR selectable marker, e.g., as described in R. J. Kaufman and P. A. Sharp, 1982 Mol. Biol. 159:601-621, NSO myeloma cells, COS cells and SP2 cells. Another expression system is the GS gene expression system shown in WO 87/04462, WO 89/01036 and EP 338,841. To produce antibodies, expression vectors encoding antibody genes are introduced into mammalian host cells, and the host cells are cultured for a period of time sufficient to allow for expression of the antibody in the host cells or secretion of the antibody into the culture medium in which the host cells are grown. Antibodies can be recovered from the culture medium using standard protein purification methods.

Standard assays to evaluate the binding ability of the antibodies toward the target of various species are known in the art, including for example, ELISAs, western blots and RIAs. The binding kinetics (e.g., binding affinity) of the antibodies also can be assessed by standard assays known in the art, such as by Biacore analysis.

General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size such as goats, dogs, sheep, mice, or camels are immunized by administration of an amount of immunogen, such as the intact protein or a portion thereof containing an epitope that is specific to the tissue or body fluid and that is indicative of a disease state, effective to produce an immune response. An exemplary protocol is as follows. The animal is subcutaneously injected in the back with 100 micrograms to 100 milligrams of antigen, dependent on the size of the animal, followed three weeks later with an intraperitoneal injection of 100 micrograms to 100 milligrams of immunogen with adjuvant dependent on the size of the animal, for example Freund's complete adjuvant. Additional intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the dilution having a detectable activity. The antibodies are purified, for example, by affinity purification on columns containing hepatic cells.

The technique of in vitro immunization of human lymphocytes is used to generate monoclonal antibodies. Techniques for in vitro immunization of human lymphocytes are well known to those skilled in the art. See, e.g., Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies. Any antibody or fragment thereof having affinity and specific for the epitope that is specific to the tissue or body fluid and that is indicative of a disease state is within the scope of the invention provided herein.

Further description of antibodies and methodologies for antibody production is shown in Shulok et al. (International patent application number PCT/US2007/000777), the contents of which are incorporated by reference herein in their entirety.

Antibodies of the invention may be used for diagnosing a disease in a subject. Antibodies of the invention are contacted to a tissue or body fluid in order to bind an epitope that is specific to the tissue or body fluid and that is indicative of a disease state. A tissue is a mass of connected cells and/or extracellular matrix material, e.g. skin tissue, nasal passage tissue, CNS tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to urine, sputum, stool, mucous, saliva, blood, plasma, serum, serum derivatives, bile, phlegm, sweat, amniotic fluid, mammary fluid, and cerebrospinal fluid (CSF), such as lumbar or ventricular CSF. A sample may also be a fine needle aspirate or biopsied tissue. A sample also may be media containing cells or biological material. In particular embodiments, the sample is a urine sample.

Antibodies of the invention exploit the principle that antibodies binding specifically to epitopes on antigens in biological fluids or tissues. An epitope refers to a specific part of an antigen that is recognized by an antibody. Recognition can be mediated by the primary sequence of a region of an antigen or the secondary, tertiary, or quaternary structure of a region of an antigen. By binding to an epitope that is specific to a tissue or body fluid and that is indicative of a disease state, antibodies of the invention may be used for diagnosis of a disease state by simple detection of the presence of the epitope in the tissue or body fluid, in contrast to diagnostic tests that rely on a concentration of a biomarker in the sample.

The antibody/antigen complex is detected to detect a disease in a patient. Generally, two strategies used for detection of epitopes on antigens in body fluids or tissues, a direct method and an indirect method. The direct method is a one-step staining method, and involves a labeled antibody (e.g. FITC conjugated antiserum) reacting directly with the antigen in a body fluid or tissue sample. The indirect method involves an unlabeled primary antibody that reacts with the body fluid or tissue antigen, and a labeled secondary antibody that reacts with the primary antibody. In either the direct or the indirect method, the antibody is labeled. Labels can include radioactive labels, fluorescent labels, hapten labels such as, biotin, or an enzyme such as horse radish peroxidase or alkaline phosphatase. Methods of conducting these assays are well known in the art. See for example, Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, Calif.).

Antibodies of the invention can be used to diagnose any disease that involves an epitope that is specific to a tissue or body fluid and that is indicative of a disease state, e.g., cancers, autoimmune diseases (e.g, systemic lupus erythematosus lupus as shown in Keene (U.S. Pat. No. 4,751,181) and Yoo (US 2002/0192723) or Crohn's disease as shown in Niwa et al. (US 2009/0170115), the contents of each of which is hereby incorporated by reference), Alzheimer's disease (as shown in Voorheis et al. (U.S. Pat. No. 5,492,812) and Wasco et al. (U.S. Pat. No. 5,891,991), the contents of each of which is hereby incorporated by reference), and diseases associated with particular organs (e.g., kidney disorders such as acute pyelonephritis and acute tubular necrosis and those shown in Ueda (Proc. Natl. Acad. Sci., Vol. 78: 5122-5126, 1981), and Thompson et al. (U.S. Pat. No. 4,731,326), the contents of each of which is hereby incorporated by reference).

In certain embodiments, the disease is cancer. Biomarkers that have been associated with types of cancers are well known in the art. Antibodies of the invention bind to an epitope of these biomarkers that are present only in particular tissues and body fluids, and the detection of these epitopes in the particular tissues and/or body fluids indicates the presence of a specific cancer in the subject.

Biomarkers associated with development of breast cancer are shown in Erlander et al. (U.S. Pat. No. 7,504,214), Dai et al. (U.S. Pat. Nos. 7,514,209 and 7,171,311), Baker et al. (U.S. Pat. No. 7,056,674 and U.S. Pat. No. 7,081,340), Erlander et al. (US 2009/0092973). The contents of the patent application and each of these patents are incorporated by reference herein in their entirety. Exemplary biomarkers that have been associated with breast cancer include: ErbB2 (Her2); ESR1; BRCA1; BRCA2; p53; mdm2; cyclin1; p2′7; B_Catenin; BAG1; BIN1; BUB1; C20_orf1; CCNB1; CCNE2; CDC20; CDH1; CEGP1; CIAP1; cMYC; CTSL2; DKFZp586M07; DRS; EpCAM; EstR1; FOXM1; GRB7; GSTM1; GSTM3; HER2; HNRPAB; ID1; IGF1R; ITGA7; Ki_{—}67; KNSL2; LMNB1; MCM2; MELK; MMP12; MMP9; MYBL2; NEK2; NME1; NPD009; PCNA; PR; PREP; PTTG1; RPLPO; Src; STK15; STMY3; SURV; TFRC; TOP2A; and TS. Antibodies of the invention bind to an epitope of these biomarkers that is present only in breast tissue and/or mammary fluid (e.g., breast milk), in which the presence of any one of the above epitopes in the breast tissue and/or the mammary fluid is indicative of breast cancer in the subject.

Biomarkers associated with development of cervical cancer are shown in Patel (U.S. Pat. No. 7,300,765), Pardee et al. (U.S. Pat. No. 7,153,700), Kim (U.S. Pat. No. 6,905,844), Roberts et al. (U.S. Pat. No. 6,316,208), Schlegel (US 2008/0113340), Kwok et al. (US 2008/0044828), Fisher et al. (US 2005/0260566), Sastry et al. (US 2005/0048467), Lai (US 2008/0311570) and Van Der Zee et al. (US 2009/0023137). The contents of each of the articles, patents, and patent applications are incorporated by reference herein in their entirety. Exemplary biomarkers that have been associated with cervical cancer include: SC6; SIX1; human cervical cancer 2 protooncogene (HCCR-2); p27; virus oncogene E6; virus oncogene E7; p16^{INK4A}; Mcm proteins (such as Mcm5); Cdc proteins; topoisomerase 2 alpha; PCNA; Ki-67; Cyclin E; p-53; PAI1; DAP-kinase; ESR1; APC; TIMP-3; RAR-β; CALCA; TSLC1; TIMP-2; DcR1; CUDR; DcR2; BRCA1; p15; MSH2; RassflA; MLH1; MGMT; SOX1; PAX1; LMX1A; NKX6-1; WT1; ONECUT1; SPAG9; and Rb (retinoblastoma) proteins. Antibodies of the invention bind to an epitope of these biomarkers that is present only in cervical tissue and/or cervical fluid (e.g., cervical mucus), in which the presence of any one of the above epitopes in the cervical tissue and/or cervical fluid is indicative of cervical cancer in the subject.

Biomarkers associated with development of vaginal cancer are shown in Giordano (U.S. Pat. No. 5,840,506), Kruk (US 2008/0009005), Hellman et al. (Br J. Cancer. 100(8):1303-1314, 2009). The contents of each of the articles, patents, and patent applications are incorporated by reference herein in their entirety. Exemplary biomarkers that have been associated with vaginal cancer include: pRb2/p130 and Bcl-2. Antibodies of the invention bind to an epitope of these biomarkers that is present only in vaginal tissue and/or vaginal fluid (e.g., vaginal mucus), in which the presence of any one of the above epitopes in the vaginal tissue and/or vaginal fluid is indicative of vaginal cancer in the subject.

Biomarkers associated with development of brain cancers (e.g., glioma, cerebellum, medulloblastoma, astrocytoma, ependymoma, glioblastoma) are shown in D'Andrea (US 2009/0081237), Murphy et al. (US 2006/0269558), Gibson et al. (US 2006/0281089), and Zetter et al. (US 2006/0160762). The contents of each of the articles and patent applications are incorporated by reference herein in their entirety. Exemplary biomarkers that have been associated with brain cancers include: epidermal growth factor receptor (EGFR); phosphorylated PKB/Akt; EGFRvIII; FANCI; Nr-CAM; antizyme inhibitor (AZI); BNIP3; and miRNA-21. Antibodies of the invention bind to an epitope of these biomarkers that is present only in brain tissue and/or cerebrospinal fluid, in which the presence of any one of the above epitopes in the brain tissue and/or cerebrospinal fluid is indicative of brain cancer in the subject.

Biomarkers associated with development of renal cancer are shown in Patel (U.S. Pat. No. 7,300,765), Soyupak et al. (U.S. Pat. No. 7,482,129), Sahin et al. (U.S. Pat. No. 7,527,933), Price et al. (U.S. Pat. No. 7,229,770), Raitano (U.S. Pat. No. 7,507,541), and Becker et al. (US 2007/0292869). The contents of each of the articles, patents, and patent applications are incorporated by reference herein in their entirety. Exemplary biomarkers that have been associated with renal cancers include: SC6; 36P6D5; IMP3; serum amyloid alpha; YKL-40; SC6; and carbonic anhydrase IX (CA IX). Antibodies of the invention bind to an epitope of these biomarkers that is present only in renal tissue and/or renal fluid (e.g., renal interstitial fluid), in which the presence of any one of the above epitopes in the renal tissue and/or renal fluid is indicative of renal cancer in the subject.

Biomarkers associated with development of hepatic cancers (e.g., hepatocellular carcinoma) are shown in Horne et al. (U.S. Pat. No. 6,974,667), Yuan et al. (U.S. Pat. No. 6,897,018), Hanausek-Walaszek et al. (U.S. Pat. No. 5,310,653), and Liew et al. (US 2005/0152908). The contents of each of the articles, patents, and patent applications are incorporated by reference herein in their entirety. Exemplary biomarkers that have been associated with hepatic cancers include: Tetraspan NET-6 protein; collagen, type V, alpha; glypican 3; pituitary tumor-transforming gene 1 (PTTG1); Galectin 3; solute carrier family 2, member 3, or glucose transporter 3 (GLUT3); metallothionein 1L; CYP2A6; claudin 4; serine protease inhibitor, Kazal type I (SPINK1); DLC-1; AFP; HSP70; CAP2; glypican 3; glutamine synthetase; AFP; AST and CEA. Antibodies of the invention bind to an epitope of these biomarkers that is present only in hepatic tissue and/or hepatic fluid, in which the presence of any one of the above epitopes in the hepatic tissue and/or hepatic fluid is indicative of hepatic cancer in the subject.

Biomarkers associated with development of gastric, gastrointestinal, and/or esophageal cancers are shown in Chang et al. (U.S. Pat. No. 7,507,532), Bae et al. (U.S. Pat. No. 7,368,255), Muramatsu et al. (U.S. Pat. No. 7,090,983), Sahin et al. (U.S. Pat. No. 7,527,933), Chow et al. (US 2008/0138806), Waldman et al. (US 2005/0100895), Goldenring (US 2008/0057514), An et al. (US 2007/0259368), Guilford et al. (US 2007/0184439), Wirtz et al. (US 2004/0018525), Filella et al. (Acta Oncol. 33(7):747-751, 1994), Waldman et al. (U.S. Pat. No. 6,767,704), and Lipkin et al. (Cancer Research, 48:235-245, 1988). The contents of each of the articles, patents, and patent applications are incorporated by reference herein in their entirety. Exemplary biomarkers that have been associated with gastric, gastrointestinal, and/or esophageal cancers include: MH15 (Hn1L); RUNX3; midkine; Chromogranin A (CHGA); Thy-1 cell surface antigen (THY1); IPO-38; CEA; CA 19.9; GroES; TAG-72; TGM3; HE4; LGALS3; IL1RN; TRIP13; FIGNL1; CRIP1; S100A4; EXOSC8; EXPI; CRCA-1; BRRN1; NELF; EREG; TMEM40; TMEM109; and guanylin cyclase C. Antibodies of the invention bind to an epitope of these biomarkers that is present only in gastric, gastrointestinal, and/or esophageal tissue and/or gastrointestinal fluid, in which the presence of any one of the above epitopes in the gastric, gastrointestinal, and/or esophageal tissue and/or gastrointestinal fluid is indicative of gastric, gastrointestinal, and/or esophageal cancer in the subject.

Biomarkers associated with development of ovarian cancer are shown in Podust et al. (U.S. Pat. No. 7,510,842), Wang (U.S. Pat. No. 7,348,142), O'Brien et al. (U.S. Pat. Nos. 7,291,462, 6,942,978, 6,316,213, 6,294,344, and 6,268,165), Ganetta (U.S. Pat. No. 7,078,180), Malinowski et al. (US 2009/0087849), Beyer et al. (US 2009/0081685), Fischer et al. (US 2009/0075307), Mansfield et al. (US 2009/0004687), Livingston et al. (US 2008/0286199), Farias-Eisner et al. (US 2008/0038754), Ahmed et al. (US 2007/0053896), Giordano (U.S. Pat. No. 5,840,506), and Tchagang et al. (Mol Cancer Ther, 7:27-37, 2008). The contents of each of the articles, patents, and patent applications are incorporated by reference herein in their entirety. Exemplary biomarkers that have been associated with ovarian cancer include: hepcidin; tumor antigen-derived gene (TADG-15); TADG-12; TADG-14; ZEB; PUMP-1; stratum corneum chymotrytic enzyme (SCCE); NES-1; μPA; PAI-2; cathepsin B; cathepsin L; ERCC5; MMP-2; pRb2/p130 gene; matrix metalloproteinase-7 (MMP-7); progesterone-associated endometrial protein (PALP); cancer antigen 125 (CA125); CTAP3; human epididymis 4 (HL4); plasminogen activator urokinase receptor (PLAUR); MUC-1; FGF-2; cSHMT; Tbx3; utrophin; SLPI; osteopontin (SSP1); mesothelin (MSLN); SPON1; interleukin-7; folate receptor 1; and claudin 3. Antibodies of the invention bind to an epitope of these biomarkers that is present only in ovarian tissue and/or ovarian fluid, in which the presence of any one of the above epitopes in the ovarian tissue and/or ovarian fluid is indicative of ovarian cancer in the subject.

Biomarkers associated with development of head-and-neck and thyroid cancers are shown in Sidransky et al. (U.S. Pat. No. 7,378,233), Skolnick et al. (U.S. Pat. No. 5,989,815), Budiman et al. (US 2009/0075265), Hasina et al. (Cancer Research, 63:555-559, 2003), Kebebew et al. (US 2008/0280302), and Ralhan (Mol Cell Proteomics, 7(6):1162-1173, 2008). The contents of each of the articles, patents, and patent applications are incorporated by reference herein in their entirety. Exemplary biomarkers that have been associated with head-and-neck and thyroid cancers include: BRAF; Multiple Tumor Suppressor (MTS); PAI-2; stratifin; YWHAZ; S100-A2; S100-A7 (psoriasin); S100-All (calgizarrin); prothymosin alpha (PTHA); L-lactate dehydrogenase A chain; glutathione S-transferase Pi; APC-binding protein EB1; fascin; peroxiredoxin2; carbonic anhydrase I; flavin reductase; histone H3; ECM1; TMPRSS4; ANGPT2; T1MP1; LOXL4; p53; IL-6; EGFR; Ku70; GST-pi; and polybromo-1D. Antibodies of the invention bind to an epitope of these biomarkers that is present only in head-and-neck or thyroid tissue and/or head-and-neck or thyroid fluid, in which the presence of any one of the above epitopes in the head-and-neck or thyroid tissue and/or head-and-neck or thyroid fluid is indicative of head-and-neck or thyroid tissue cancer in the subject.

Biomarkers associated with development of colorectal cancers are shown in Raitano et al. (U.S. Pat. No. 7,507,541), Reinhard et al. (U.S. Pat. No. 7,501,244), Waldman et al. (U.S. Pat. No. 7,479,376); Schleyer et al. (U.S. Pat. No. 7,198,899); Reed (U.S. Pat. No. 7,163,801), Robbins et al. (U.S. Pat. No. 7,022,472), Mack et al. (U.S. Pat. No. 6,682,890), Tabiti et al. (U.S. Pat. No. 5,888,746), Budiman et al. (US 2009/0098542), Karl (US 2009/0075311), Arjol et al. (US 2008/0286801), Lee et al. (US 2008/0206756), Mori et al. (US 2008/0081333), Wang et al. (US 2008/0058432), Belacel et al. (US 2008/0050723), Stedronsky et al. (US 2008/0020940), An et al. (US 2006/0234254), Eveleigh et al. (US 2004/0146921), and Yeatman et al. (US 2006/0195269). The contents of each of the articles, patents, and patent applications are incorporated by reference herein in their entirety. Exemplary biomarkers that have been associated with colorectal cancers include: 36P6D5; TTK; CDX2; NRG4; TUCAN; hMLH1; hMSH2; M2-PK; CGA7; CJA8; PTP.alpha.; APC; p53; Ki-ras; complement C3a des-arg; alpha1-antitrypsin; transferrin; MMP-11; CA-19-9; TPA; TPS; TIMP-1; C10orf3; carcinoembryonic antigen (CEA); a soluble fragment of cytokeratin 19 (CYFRA 21-1); TAC1; carbohydrate antigen 724 (CA72-4); nicotinamide N-methyltransferase (NNMT); pyrroline-5-carboxylate reductase (PROC); S-adenosylhomocysteine hydrolase (SAHH); IBABP-L polypeptide; and Septin 9. Antibodies of the invention bind to an epitope of these biomarkers that is present only in colon and/or rectal tissue, in which the presence of any one of the above epitopes in the colon and/or rectal tissue is indicative of colorectal cancer in the subject.

Biomarkers associated with development of prostate cancer are shown in Sidransky (U.S. Pat. No. 7,524,633), Platica (U.S. Pat. No. 7,510,707), Salceda et al. (U.S. Pat. No. 7,432,064 and U.S. Pat. No. 7,364,862), Siegler et al. (U.S. Pat. No. 7,361,474), Wang (U.S. Pat. No. 7,348,142), Ali et al. (U.S. Pat. No. 7,326,529), Price et al. (U.S. Pat. No. 7,229,770), O'Brien et al. (U.S. Pat. No. 7,291,462), Golub et al. (U.S. Pat. No. 6,949,342), Ogden et al. (U.S. Pat. No. 6,841,350), An et al. (U.S. Pat. No. 6,171,796), Bergan et al. (US 2009/0124569), Bhowmick (US 2009/0017463), Srivastava et al. (US 2008/0269157), Chinnaiyan et al. (US 2008/0222741), Thaxton et al. (US 2008/0181850), Dahary et al. (US 2008/0014590), Diamandis et al. (US 2006/0269971), Rubin et al. (US 2006/0234259), Einstein et al. (US 2006/0115821), Paris et al. (US 2006/0110759), Condon-Cardo (US 2004/0053247), and Ritchie et al. (US 2009/0127454). The contents of each of the articles, patents, and patent applications are incorporated by reference herein in their entirety. Exemplary biomarkers that have been associated with prostate cancer include: PSA; GSTP1; PAR; CSG; MIF; TADG-15; p53; YKL-40; ZEB; HOXC6; Pax 2; prostate-specific transglutaminase; cytokeratin 15; MEK4; MIP1-β; fractalkine; IL-15; ERGS; EZH2; EPC1; EPC2; NLGN-4Y; kallikrein 11; ABP280 (FLNA); AMACR; AR; BM28; BUB3; CaMKK; CASPASE3; CDK7; DYNAMIN; E2F1; E-CADHERIN; EXPORTIN; EZH2; FAS; GAS7; GS28; ICBP90; ITGA5; JAGGED1; JAM1; KANADAPTIN; KLF6; KRIP1; LAP2; MCAM; MIB1 (MKI67); MTA1; MUC1; MYOSIN-VI; P27; P63; P27; PAXILLIN; PLCLN; PSA(KLK3); RAB27; RBBP; RIN1; SAPKα; TPD52; XIAP; ZAG; and semenogelin II. Antibodies of the invention bind to an epitope of these biomarkers that is present only in prostate tissue, in which the presence of any one of the above epitopes in the prostate tissue is indicative of prostate cancer in the subject.

Biomarkers associated with development of pancreatic cancer are shown in Sahin et al. (U.S. Pat. No. 7,527,933), Rataino et al. (U.S. Pat. No. 7,507,541), Schleyer et al. (U.S. Pat. No. 7,476,506), Domon et al. (U.S. Pat. No. 7,473,531), McCaffey et al. (U.S. Pat. No. 7,358,231), Price et al. (U.S. Pat. No. 7,229,770), Chan et al. (US 2005/0095611), Mitchl et al. (US 2006/0258841), and Faca et al. (PLoS Med 5(6):e123, 2008). The contents of each of the articles, patents, and patent applications are incorporated by reference herein in their entirety. Exemplary biomarkers that have been associated with pancreatic cancer include: CA19.9; 36P6D5; NRG4; ASCT2; CCR7; 3C4-Ag; KLK11; Fibrinogen γ; and YKL40. Antibodies of the invention bind to an epitope of these biomarkers that is present only in pancreatic tissue, in which the presence of any one of the above epitopes in the pancreatic tissue is indicative of pancreatic cancer in the subject.

Biomarkers associated with development of lung cancer are shown in Sahin et al. (U.S. Pat. No. 7,527,933), Hutteman (U.S. Pat. No. 7,473,530), Bae et al. (U.S. Pat. No. 7,368,255), Wang (U.S. Pat. No. 7,348,142), Nacht et al. (U.S. Pat. No. 7,332,590), Gure et al. (U.S. Pat. No. 7,314,721), Patel (U.S. Pat. No. 7,300,765), Price et al. (U.S. Pat. No. 7,229,770), O'Brien et al. (U.S. Pat. No. 7,291,462 and U.S. Pat. No. 6,316,213), Muramatsu et al. (U.S. Pat. No. 7,090,983), Carson et al. (U.S. Pat. No. 6,576,420), Giordano (U.S. Pat. No. 5,840,506), Guo (US 2009/0062144), Tsao et al. (US 2008/0176236), Nakamura et al. (US 2008/0050378), Raponi et al. (US 2006/0252057), Yip et al. (US 2006/0223127), Pollock et al. (US 2006/0046257), Moon et al. (US 2003/0224509), and Budiman et al. (US 2009/0098543). The contents of each of the articles, patents, and patent applications are incorporated by reference herein in their entirety. Exemplary biomarkers that have been associated with lung cancer include: COX-2; COX4-2; RUNX3; aldoketoreductase family 1, member B 10; peroxiredoxin 1 (PRDX1); TNF receptor superfamily member 18; small proline-rich protein 3 (SPRR3); SOX1; SC6; TADG-15; YKL40; midkine; DAP-kinase; HOXA9; SCCE; STX1A; HIF1A; CCT3; HLA-DPB1; MAFK; RNF5; KIF11; GHSR1b; NTSR1; FOXM1; and PUMP-1. Antibodies of the invention bind to an epitope of these biomarkers that is present only in lung tissue and/or lung fluid (e.g., pleural fluid), in which the presence of any one of the above epitopes in the lung tissue and/or lung fluid is indicative of lung cancer in the subject.

Biomarkers associated with development of skin cancer (e.g., basal cell carcinoma, squamous cell carcinoma, and melanoma) are shown in Roberts et al. (U.S. Pat. No. 6,316,208), Polsky (U.S. Pat. No. 7,442,507), Price et al. (U.S. Pat. No. 7,229,770), Genetta (U.S. Pat. No. 7,078,180), Carson et al. (U.S. Pat. No. 6,576,420), Moses et al. (US 2008/0286811), Moses et al. (US 2008/0268473), Dooley et al. (US 2003/0232356), Chang et al. (US 2008/0274908), Alani et al. (US 2008/0118462), Wang (US 2007/0154889), and Zetter et al. (US 2008/0064047). The contents of each of the articles, patents, and patent applications are incorporated by reference herein in their entirety. Exemplary biomarkers that have been associated with skin cancer include: p27; Cyr61; ADAMTS-7; Cystatin B; Chaperonin 10; Profilin; BRAF; YKL-40; DDX48; erbB3-binding protein; biliverdin reductase; PLAB; L1CAM; SAA; CRP; SOX9; MMP2; CD10; and ZEB. Antibodies of the invention bind to an epitope of these biomarkers that is present only in skin tissue, in which the presence of any one of the above epitopes in the skin tissue is indicative of skin cancer in the subject.

Biomarkers associated with development of multiple myeloma are shown in Coignet (U.S. Pat. No. 7,449,303), Shaughnessy et al. (U.S. Pat. No. 7,308,364), Seshi (U.S. Pat. No. 7,049,072), and Shaughnessy et al. (US 2008/0293578, US 2008/0234139, and US 2008/0234138). The contents of each of the articles, patents, and patent applications are incorporated by reference herein in their entirety. Exemplary biomarkers that have been associated with multiple myeloma include: JAG2; CCND1; MAF; MAFB; MMSET; CST6; RAB7L1; MAP4K3; HRASLS2; TRAIL; IG; FGL2; GNG11; MCM2; FLJ10709; TRIM13; NADSYN1; TRIM22; AGRN; CENTD2; SESN1; TM7SF2; NICKAP1; COPG; STAT3; ALOX5; APP; ABCB9; GAA; CEP55; BRCA1; ANLN; PYGL; CCNE2; ASPM; SUV39H2; CDC25A; IFIT5; ANKRA2; PHLDB1; TUBA1A; CDCA7; CDCA2; HFE; RIF1; NEIL3; SLC4A7; FXYD5; MCC; MKNK2; KLHL24; DLC1; OPN3; B3GALNT1; SPRED1; ARHGAP25; RTN2; WNT16; DEPDC1; STT3B; ECHDC2; ENPP4; SAT2; SLAMF7; MAN1C1; INTS7; ZNF600; L3 MBTL4; LAPTM4B; OSBPL10; KCNS3; THEX1. CYB5D2; UNC93B1; SIDT1; TMEM57; HIGD24; FKSG44; C14orf28; LOC387763; TncRNA; C18orf1; DCUN1D4; FANCI; ZMAT3; NOTCH1; BTG2; RAB1A; TNFRSF10B; HDLBP; RIT1; KIF2C; S100A4; MEIS1; SGOL2; CD302; COX2; C5orf34; FAM111B; C18orf54; and TP53. Antibodies of the invention bind to an epitope of these biomarkers that is present only in bone marrow and/or blood, in which the presence of any one of the above epitopes in the bone marrow and/or blood is indicative of multiple myeloma in the subject.

Biomarkers associated with development of leukemia are shown in Ando et al. (U.S. Pat. No. 7,479,371), Coignet (U.S. Pat. No. 7,479,370 and U.S. Pat. No. 7,449,303), Davi et al. (U.S. Pat. No. 7,416,851), Chiorazzi (U.S. Pat. No. 7,316,906), Seshi (U.S. Pat. No. 7,049,072), Van Baren et al. (U.S. Pat. No. 6,130,052), Taniguchi (U.S. Pat. No. 5,643,729), Insel et al. (US 2009/0131353), and Van Bockstaele et al. (Blood Rev. 23(1):25-47, 2009). The contents of each of the articles, patents, and patent applications are incorporated by reference herein in their entirety. Exemplary biomarkers that have been associated with leukemia include: SCGF; JAG2; LPL; ADAM29; PDE; Cryptochrome-1; CD49d; ZAP-70; PRAME; WT1; CD15; CD33; and CD38. Antibodies of the invention bind to an epitope of these biomarkers that is present only in bone marrow and/or blood, in which the presence of any one of the above epitopes in the bone marrow and/or blood is indicative of leukemia in the subject.

Biomarkers associated with development of lymphoma are shown in Ando et al. (U.S. Pat. No. 7,479,371), Levy et al. (U.S. Pat. No. 7,332,280), and Arnold (U.S. Pat. No. 5,858,655). The contents of each of the articles, patents, and patent applications are incorporated by reference herein in their entirety. Exemplary biomarkers that have been associated with lymphoma include: SCGF; LMO2; BCL6; FN1; CCND2; SCYA3; BCL2; CD79a; CD7; CD25; CD45RO; CD45RA; and PRAD1 cyclin. Antibodies of the invention bind to an epitope of these biomarkers that is present only in bone marrow and/or blood, in which the presence of any one of the above epitopes in the bone marrow and/or blood is indicative of leukemia in the subject.

Biomarkers associated with development of bladder cancer are shown in Price et al. (U.S. Pat. No. 7,229,770), Orntoft (U.S. Pat. No. 6,936,417), Haak-Frendscho et al. (U.S. Pat. No. 6,008,003), Feinstein et al. (U.S. Pat. No. 6,998,232), Elting et al. (US 2008/0311604), and Wewer et al. (2009/0029372). The contents of each of the patent applications and each of these patents are incorporated by reference herein in their entirety. Exemplary biomarkers that have been associated with bladder cancer include: NT-3; NGF; GDNF; YKL-40; p53; pRB; p21; p27; cyclin E1; Ki67; Fas; urothelial carcinoma-associated 1; human chorionic gonadotropin beta type II; insulin-like growth factor-binding protein 7; sorting nexin 16; chondroitin sulfate proteoglycan 6; cathepsin D; chromodomain helicase DNA-binding protein 2; nell-like 2; tumor necrosis factor receptor superfamily member 7; cytokeratin 18 (CK18); ADAMS; ADAM10; ADAM12; MMP-2; MMP-9; KAI1; and bladder tumor fibronectin (BTF). Antibodies of the invention bind to an epitope of these biomarkers that is present only in bladder tissue and/or urine, in which the presence of any one of the above epitopes in the bladder tissue and/or urine is indicative of breast cancer in the subject.

In certain embodiments, the epitope is an epitope on a ADAM protein that is present in urine from a subject, in which presence of the epitope is indicative of bladder cancer. In a particular embodiment, the epitope is an epitope on an ADAM12 protein that is present in urine from a subject, in which presence of the epitope is indicative of bladder cancer. A disintegrin and metalloproteinases (ADAMs) are a gene family of proteins that belong to the metzincin-superfamily of zinc-dependent metalloproteinases that share the metalloproteinase domain with matrix metalloproteinases (MMPs). These molecules are involved in various biological events such as cell adhesion, cell fusion, cell migration, membrane protein shedding and proteolysis. The activities of ADAMs are regulated by gene expression, intracytoplasmic and pericellular regulation, activation of the zymogens and inhibition of activities by inhibitors (Edwards D, et al. Mol Asp of Med 29: 258-289, 2008, contents of which is hereby incorporated by reference).

The ADAM family is important to many control processes in development and homeostasis and therefore disregulation of the functions of ADAMs is linked to pathological states. Diseases associated with ADAM disregulation include cancer, cardiovascular disease, asthma, and Alzheimer's disease. Many ADAM species are expressed in human malignant tumors, including ADAMS, ADAM9, ADAM10, ADAM12, ADAM15, ADAM17, ADAM19, ADAM28, ADAMTS1, ADAMTS4 and ADAMTS5.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Recombinant ADAM12 Expression and Purification

Initial expression experiments showed that a majority of ADAM12 protein in the sample was found in intracellular soluble fractions and was not being secreted into media. These results were obtained using an anti-tag antibody to confirm expression and location of the protein. The next step in the process was to express 1L and test-purify the soluble fraction using anti-tag affinity purification. Western blots were run using the anti-tag antibody and confirmed the expression and purification of tagged ADAM12 from the soluble cell fraction (using 2.5 ul of each fractions/per lane). The ADAM12 still was not detected in the conditioned media (using 12.5 ul). The 5 L scale-up was undertaken and the soluble fraction only was purified, discarding all the conditioned media.

Aliquots of the soluble cell fraction (from Roopali Roy laboratory, Children's Hospital, Boston Mass.), the purified fractions from the soluble fraction (from Blue Sky Biotech, Worcester, Mass.), and the conditioned media (from Blue Sky Biotech, Worcester, Mass.), were obtained. These fractions were run on westerns using the AbCam ab28225 polyclonal ADAM12 antibody. In the western, ADAM12 was detected both in the purified fractions from the soluble fraction (using 2 ul of each fraction per lane) as well as in the conditioned media (using 30 ul). See FIG. 1.

The molecular weight of the ADAM12 detected in the fractions was consistent with the latent form of the enzyme, whereas the molecular weight of the ADAM12 found in the conditioned media was consistent with the active form. This molecular weight was also more consistent with the form of ADAM12 observed by the Moses lab (Children's Hospital, Boston's Vascular Biology Program) in their initial identification of ADAM12 as a marker of breast cancer status.

Interestingly, numerous bands were detected in the whole soluble fraction using ab28225. See FIG. 1. These results are similar to observations related to urine samples contacted with ab28225, and confirm the lack of specificity of the AbCam antibody.

### Example 2

### Immunization of Rats and Mice with Recombinant ADAM12 (from Soluble Fraction)

Four rats and five mice were immunized with the purified ADAM12 from the soluble fraction. A 28-day standard immunization protocol was followed. The mice and rats were bled before immunization (negative control) and at day 24 to test for immune reaction to ADAM12. Sera were tested in direct ELISA format against pure ADAM12 from the soluble fraction (cell paste), pure recombinant ADAM12 from conditioned media, and FHL (negative control).

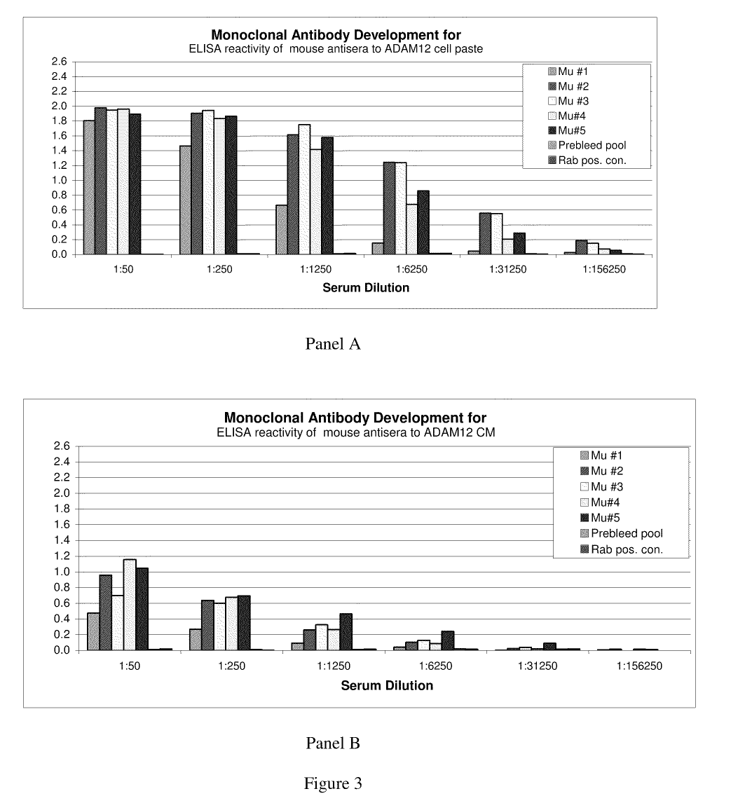

All five mice reacted to ADAM12 specifically as determined by direct ELISA. Mice 2, 3 and 5 had the highest titer against ADAM12 from the cell paste, while mice 2, 4 and 5 had the highest titer against the ADAM12 from the conditioned media. In all cases, titer to the cell paste was higher than to the conditioned media. From these data (FIG. 3 panels A and B), it was determined that mouse 2 or 5 would be the best candidates for Fusion.

All four rats reacted to ADAM12 specifically as determined by direct ELISA. Rats 2 and 3 had the highest titer. In all cases, titer to the cell paste was higher than to the conditioned media. Rat 2 was chosen to for use in sandwich ELISA format, either as a capture antibody or a detection antibody. Conditions were later optimized as described below.

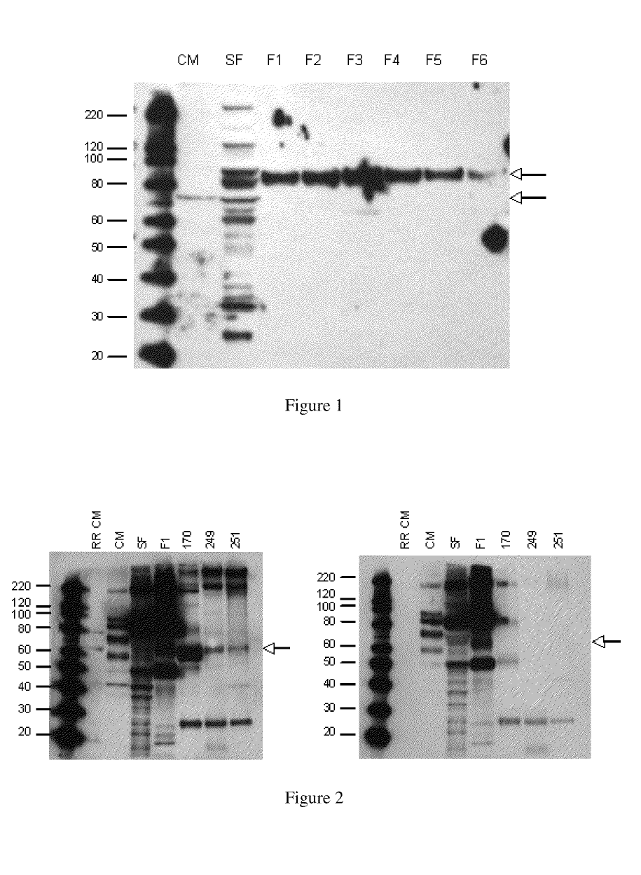

Three urine samples suspected to have elevated levels of ADAM12 were chosen for testing (one bladder cancer and two breast cancer samples). Gels were run under reducing and non-reducing conditions. Conditioned media (from Roopali Roy laboratory, Children's Hospital, Boston Mass.), conditioned media (from Blue Sky Biotech, Worcester, Mass.), soluble fraction (from Blue Sky Biotech, Worcester, Mass.) and Fraction 1 (from Blue Sky Biotech, Worcester, Mass.) were used as positive controls in each gel. Of all 5 mice, only mouse 2 reacted strongly to ADAM12 in urine. The band of interest at approximately 68 kD was detected under reducing conditions. Representative blots from mouse 2 and mouse 5 under reducing conditions are shown in FIG. 2. Based on these results, mouse 2 was chosen for going forward to fusion instead of mouse 5.

Although all four rats reacted to ADAM12 on ELISA, none reacted to the band of interest in the urine samples by western analysis. Rat 3 had some reactivity but at lower molecular weight than the band of interest. Tests using the rat 3/mouse 2 sandwich ELISA did not result in detection of ADAM12 in urine. Based on these results, it was concluded that primary screens of fusion clones using urine samples would be difficult.

### Example 3

### Fusion Phase for Hybridoma Development

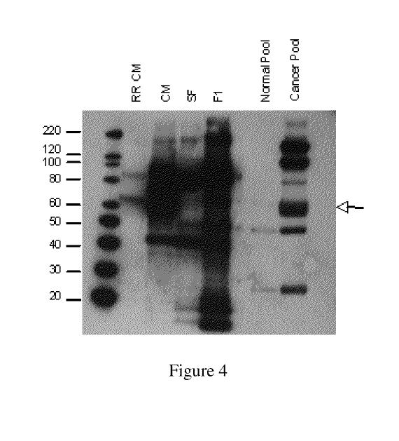

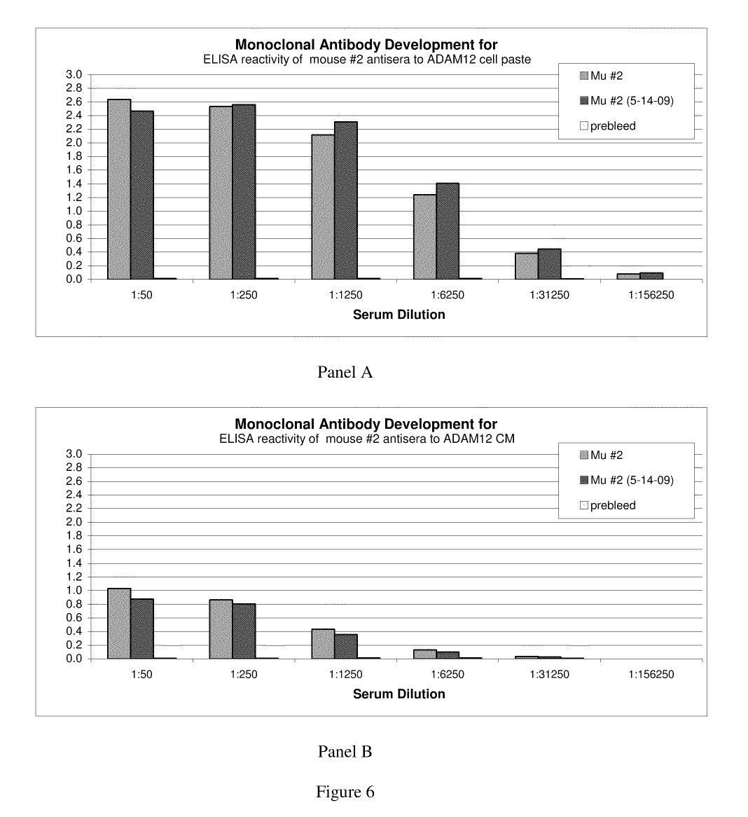

Mouse 2 proceeded to fusion. Terminal bleeds were taken for testing against urine (western) and for ELISA. Spleen and lymph nodes were taken to harvest the cells and plates were set up for growing and clone selection. ELISA results showed similar titers of the terminal bleed as previously seen for mouse 2 (FIG. 6 panels A and B). Western analysis of the normal and cancer pools showed the band of interest in the cancer pool but not in the normal pool (FIG. 4), suggesting that at time of fusion, the antibody of interest was well represented.

### Example 4

### Primary Screening

A sandwich ELISA using mouse 2 sera and rat 3 sera for capture and detection was developed. The assay did not result in detection of ADAM12 in the pool of urine from cancer patients. The results suggested that primary screens should include screening against urine samples. Thus, other detection strategies were investigated, including: direct ELISA against recombinant ADAM12 from cell paste; direct ELISA against recombinant ADAM12 from conditioned media; direct ELISA against FHL as a negative control screen; or IgG secretion analysis for identifying those clones that produce adequate amounts of antibody.

A direct ELISA against recombinant ADAM12 was investigated. The data from the direct ELISA resulted in 131 positive clones from a possible 1900. The results were as follows: 36 positive for cell paste, conditioned media and producing IgG; 38 positive for cell paste and producing IgG; 3 positive for conditioned media and producing IgG; and 54 IgG producers that did not cross react with ADAM12.

### Example 5

### Secondary Screening

Efforts to directly bind urine ADAM12 to plastic for screening were unsuccessful, and thus all positive fusion products were screened by western. The 54 IgG producers that did not cross-react with ADAM12 were tested in pools of 3, such that the total number of blots run equaled 95. Pools of normal and cancer urines were used in these western screens. Recombinant ADAM12 from the cell paste or conditioned media was used as positive controls.

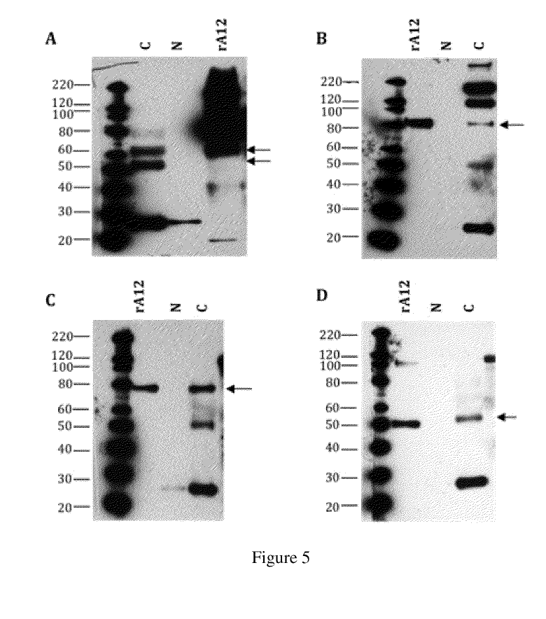

This screen resulted in the identification of two types of antibodies: those that recognized bands of ˜80 kD and greater and those that recognized bands of ˜68 kD and lower. Of the 95 blots, 4 were strongly positive for the ˜68 kD band, 4 were weakly positive for the ˜68 kDb and, 11 were strongly positive for the ˜80 kD band and 71 were positive for a band of ˜55 kd). The 80 kd) band was consistent with the unprocessed, full-length ADAM12, while the ˜68 kD band was consistent with the cleaved, active form of the enzyme. Representative blots are shown in FIG. 5.

### Example 6

### Subcloning

Four antibody candidates were chosen that recognized either high molecular weight species of ADAM12 (consistent with the latent form of the enzyme) or lower molecular weight species (consistent with the secreted form of ADAM12) from the fusion screens for subcloning and scale-up. The four antibodies were tested after scale-up for ability to detect ADAM12 in individual urine samples, including 10 bladder cancer samples, 10 breast cancer samples and 9 normal samples.

Concurrently, the four candidate antibodies were subcloned to produce pure monoclonal cell lines from each. These subclones were screened using pooled urine as previously described. Since a number of subclones had the same band recognition pattern for the bands of interest, the clones with the highest analytical sensitivity were chosen for ascites production.

### Example 7

### Results

Antibody 13G04: This antibody recognized the full-length recombinant ADAM12 at ˜80kDa. It also previously recognized bands of ˜120 and ˜240 kDa in addition to the ˜80 kDa band in the urine pools. This antibody did not recognize ADAM12 in the breast cancer samples, but had very strong reactivity against ADAM12 in the bladder cancer samples. No high molecular weight ADAM12 was detected in normal samples. The bladder cancer samples consisted of 2 T4, 2 T2, 2 T1, 2 Tis and 2 Ta. One or more bands representing ADAM12 were detected in all ten samples. Results are shown in Table 1 below.

TABLE 1

| Sample | Stage | ~80 kDa | ~120 kDa | ~240 kDa | Any Three |
|---|---|---|---|---|---|
| 1 | T4 | X | X | X | X |
| 2 | T4 | X | X | X | X |
| 3 | T2 | X | X | X | X |
| 4 | T2 | X | X | X | X |
| 5 | T1 | X | X | X | X |
| 6 | T1 | X | X | X | X |
| 7 | Tis | X | | | X |
| 8 | Tis | X | X | X | X |
| 9 | Ta | | X | X | X |
| 10 | Ta | X | X | X | X |
| | | 90% sens | 90% sens | 90% sens | 100% sens |
| | | 100% spec | 100% spec | 100% spec | 100% spec |

Antibody Fusion 07D12: This antibody recognized the full-length recombinant ADAM12 at ˜80 kDa. It also previously recognized bands of ˜120 and ˜240 kDa in addition to the ˜80 kDa band in the urine pools. This antibody recapitulated the results obtained with 13G04, but was not as sensitive as 13G04.

Antibody Fusion 05C11: This antibody recognized the secreted recombinant ADAM12 in conditioned media with a band at ˜68 kDa consistent with the active form of the enzyme. It also previously recognized bands of ˜50 and ˜68 kDa in the urine pools. This antibody had weak reactivity to ADAM12 in the breast cancer urines but strong reactivity against ADAM12 in all 10 urines from bladder cancer patients (at ˜50 kDa). Extremely weak or no reactivity was detected in normal urine, such that 100% sensitivity and 100% specificity was achieved.

Antibody Fusion 02A04: This antibody recognized the secreted recombinant ADAM12 in conditioned media with a band at ˜68 kDa consistent with the active form of the enzyme. It also previously recognized bands of ˜50 and ˜68 kDa in the urine pools. This antibody had the best reactivity to ADAM12 in the breast cancer urines. 9 out of 10 had bands at ˜68 kDa while only 2 out of 9 normal samples had similar bands. Although a band of ˜50 kDa was detected in all 10 urines from bladder cancer patients the sensitivity of this species of ADAM12 I bladder cancer urines was higher using antibody 05C11.

### Example 8

### Ascites Production and Antibody Purification

Given the results described above, a single clone from 13G04 was taken into ascites production. Ascites production and purification yielded ˜200 mg of pure antibody. Two subclones of 02A04 with some band recognition pattern differences were taken into ascites production.

What is claimed:

1. An isolated human or humanized antibody or functional fragment thereof comprising an antigen-binding region that binds to an epitope that is specific to a tissue or body fluid and wherein the epitope is indicative of a disease.

2. An isolated protein comprising an epitope that is specific to a tissue or body fluid and that is indicative of a disease.

3. An isolated human or humanized antibody or functional fragment thereof comprising an antigen-binding region that is specific for ADAM12 present in urine and that is indicative of a cancer, wherein the antibody or functional fragment thereof is capable of binding to ADAM12.

4. The antibody of claim 4, wherein the cancer is selected from the group consisting of bladder, ovarian, and breast cancer.

5. An ADAM12 protein comprising an epitope present only in urine, wherein the epitope is indicative of a cancer.

6. The ADAM12 protein of claim 5, wherein the cancer is bladder cancer or ovarian cancer.

7. A method for obtaining a tissue-specific antibody for diagnosing a disease, the method comprising:
producing a panel of antibodies in response to a presenting antigen;
exposing said panel to a tissue or body fluid sample obtained from a patient; and
selecting members of said panel that bind to an epitope specific to said sample and that is indicative of the presence of disease.

8. A method for disease diagnosis, the method comprising:
obtaining a plurality of antibodies produced by antigenic challenge in a first organism;
screening said plurality against a sample obtained from a second organism; and
selecting a member of said plurality that binds to an epitope present in said sample, wherein said epitope is specific to the sample and is indicative of the presence of a disease state in the organism from whom the sample was obtained.

9. The method of claim 7 or 8, further comprising the step of diagnosing said disease.

10. The method of claim 9, wherein the disease is cancer.

11. The method of claim 10, wherein the cancer is selected from the group consisting of brain, kidney, liver, adrenal gland, bladder, cervix, breast, stomach, ovaries, esophagus, neck, head, skin, colon, rectum, prostate, pancreas, liver, lung, vagina, thyroid, sarcomas, glioblastomas, multiple myeloma, blood, or gastrointestinal.

12. The method of claim 7 or 8, wherein the body fluid sample is selected from the group consisting of urine, sputum, stool, blood, mucus, and saliva.

13. The method of claim 7 or 8, wherein the epitope presents on a protease.

14. The method of claim 13, wherein the protease is a metalloproteinase.

15. The method of claim 14, wherein the metalloproteinase is ADAM12.

16. A method of diagnosing a cancer in a subject, the method comprising:
obtaining a urine sample from a subject; and
contacting an antibody to the urine sample to detect an ADAM12 epitope that is specific to urine, wherein presence of the epitope is indicative of a cancer.

17. The method of claim 16 wherein the cancer is chosen from the group consisting of bladder, ovarian, and breast cancer.

18. The method of claim 16 wherein the antibody is a polyclonal antibody or functional fragment thereof.

19. The method of claim 16 wherein the antibody is a monoclonal antibody or functional fragment thereof.

20. A method of diagnosing a disease in a subject, the method comprising:
obtaining a tissue or body fluid from a subject;
contacting an antibody to the tissue or body fluid in order to detect an epitope that is specific to the tissue or body fluid and that is indicative of a disease state; and
diagnosing the disease based on results of the contacting step.

21. A method of diagnosing a cancer in a subject, the method comprising:
obtaining a urine sample from a subject; and
contacting an antibody to the urine sample to detect an ADAM12 epitope that is specific to urine, wherein presence of the epitope is indicative of a cancer.
